(19) United States
(12) Patent Application Publication
(10) Pub. No.: US 2021/0045720 A1
(43) Pub. Date: Feb. 18, 2021

(54) COVERED COIL SHEATH FOR BIOPSY NEEDLE, BIOPSY NEEDLE ASSEMBLY, AND METHOD OF FORMING COVERED COIL SHEATH

(71) Applicant: HOYA CORPORATION, Tokyo (JP)

(72) Inventors: Christopher KADAMUS, West Roxbury, MA (US); Robb Morse GAVALIS, Westborough, MA (US)

(73) Assignee: HOYA CORPORATION, Tokyo (JP)

(21) Appl. No.: 16/980,360

(22) PCT Filed: Mar. 13, 2019
(86) PCT No.: PCT/IB2019/000615

§ 371 Date: Sep. 11, 2020

Related U.S. Application Data
(60) Provisional application No. 62/643,082, filed on Mar. 14, 2018.

(51) Int. Cl. A61B 10/02

(52) U.S. Cl. CPC A61B 10/0233

(57) ABSTRACT

A sheath comprises a coil of wire, the coil having an interior that defines a lumen; a distal anchor secured to the coil near a distal end of the coil and having a barb that extends away from an axis of the coil; a proximal anchor secured to the coil near a proximal end of the coil and having a barb that extends away from the axis of the coil; and a polymer heat-shrink sleeve. The polymer heat-shrink sleeve is coaxial with the coil, circumferentially encloses and is shrunken onto the coil over a length from the distal anchor to the proximal anchor, and extends over the barb of the distal anchor and the barb of the proximal anchor.

### CROSS-REFERENCE TO RELATED APPLICATIONS

This application claims priority to U.S. Provisional Patent Application No. 62/643,082, filed Mar. 14, 2018, which is incorporated by reference herein in its entirety.

### BACKGROUND

### Field

The present disclosure generally relates to surgical devices, methods of fabrication of surgical devices, and methods of use of surgical devices. More particularly, and without limitation, the disclosed embodiments relate to devices, systems, and methods for endoscopic tissue collection.

### Background

Fine needle biopsy (FNB) and fine needle aspiration (FNA) are commonly employed during Endoscopic Ultrasound (EUS) procedures to acquire tissue samples that would have been collected through open surgical or percutaneous techniques in the past. For example, endoscopic ultrasound-guided fine needle biopsy (EUS-FNB) techniques, such as endoscopic ultrasound-guided fine needle aspiration (EUS-FNA), have become effective and minimally invasive diagnostic sampling methods in patients with gastrointestinal or pancreatic lesions. EUS-FNA and EUS-FNB combine endoscopic visualization with ultrasound imaging and a sampling device. These techniques allow physicians to use traditional endoscopic visualization to guide their way through a tract of the body (e.g., the gastrointestinal tract) and use ultrasound imaging to provide images of organs and structures beyond the wall of the tract to guide sampling of a desired location. Then, an elongated biopsy needle device is passed through the biopsy channel of the endoscope and is visualized ultrasonically as it penetrates to the desired sampling location to collect a tissue or biological liquid sample.

### SUMMARY

A sheath according to a general configuration comprises a coil of wire, the coil having an interior that defines a lumen; a distal anchor secured to the coil near a distal end of the coil and having a barb that extends away from an axis of the coil; a proximal anchor secured to the coil near a proximal end of the coil and having a barb that extends away from the axis of the coil; and a polymer heat-shrink sleeve. The polymer heat-shrink sleeve is coaxial with the coil, circumferentially encloses and is shrunken onto the coil over a length from the distal anchor to the proximal anchor, and extends over the barb of the distal anchor and the barb of the proximal anchor. In this sheath, in a first plane that includes the axis of the coil, an angle between a distal direction of the axis and a distal surface of the barb of the distal anchor is not more than ninety degrees. In this sheath, in a second plane that includes the axis of the coil, an angle between a proximal direction of the axis and a proximal surface of the barb of the proximal anchor is not more than ninety degrees.

A biopsy needle device according to a general configuration comprises a sheath as described in the preceding paragraph and a needle moveably disposed within the lumen.

A method of forming a sheath according to a general configuration comprises securing a distal anchor near a distal end of a coil of wire, the coil having an interior that defines a lumen, the distal anchor having a barb that extends away from an axis of the coil; securing a proximal anchor near a proximal end of the coil, the proximal anchor having a barb that extends away from the axis of the coil; and applying heat to shrink a polymer heat-shrink sleeve to be coaxial with and shrunken onto the coil. In this method, the shrunken sleeve circumferentially encloses the coil over a length from the distal anchor to the proximal anchor and extends over the barb of the distal anchor and the barb of the proximal anchor. In this method, in a first plane that includes the axis of the coil, an angle between a distal direction of the axis and a distal surface of the barb of the distal anchor is not more than ninety degrees. In this method, in a second plane that includes the axis of the coil, an angle between a proximal direction of the axis and a proximal surface of the barb of the proximal anchor is not more than ninety degrees.

### BRIEF DESCRIPTION OF THE DRAWINGS

FIG. 1A is a photograph of an FNA needle with a solid polymer (PEEK) sheath.

FIG. 1B is a photograph of an FNA needle with a coated wire coil sheath.

FIG. 1C is a photograph of an FNA needle with an uncoated stainless-steel coil sheath.

FIG. 2A is a schematic drawing of sheath 10 in cross-section.

FIG. 2B shows cross-sections of wire stocks of alternative, non-rectangular cross-sectional shape that may be wound to form other implementations of spring guide 100.

FIG. 3A shows a cross-section of spring guide 100.

FIG. 3B shows a side view of spring guide 100.

FIG. 4A shows a side view of distal anchor 300.

FIG. 4B shows a side view of proximal anchor 400.

FIG. 4C shows an end view of an implementation 310 of distal anchor 300.

FIG. 5A shows a cross-sectional view of distal anchor 300 and proximal anchor 400 secured to spring guide 100.

FIG. 5B shows a side view of distal anchor 300 and proximal anchor 400 secured to spring guide 100.

FIG. 6A shows profiles of alternative implementations of protrusions of anchors 300 and 400.

FIG. 6B shows a cross-section (left) and a plan view (right) of a further example of a protrusion of anchor 300 and/or 400.

FIG. 7A shows a perspective view of an implementation 110 of spring guide 100.

FIG. 7B shows a cross-sectional view of an implementation 310 of distal anchor 300 secured to spring guide 110.

FIG. 8A is a side view of an implementation 350 of distal anchor 300.

FIG. 8B is a perspective view of distal anchor 350.

FIG. 8C is a cross-sectional view of distal anchor 350.

FIG. 9A shows a perspective view of an implementation 120 of spring guide 100.

FIG. 9B shows a perspective view of distal anchor 350 mounted to spring guide 120.

FIG. 9C shows a cross-sectional view of distal anchor 350 mounted to spring guide 120.

FIG. 10 is a cross-sectional view of an implementation 20 of sheath 10.

FIG. 11A is a side view of an implementation 450 of proximal anchor 400.

FIG. 11B is a perspective view of proximal anchor 450.

FIG. 11C is a cross-sectional view of proximal anchor 450.

FIG. 12A is a perspective view of a distal end of sheath 20.

FIG. 12B is a perspective view of a proximal end of an implementation 30 of sheath 20.

### DETAILED DESCRIPTION

The disclosed embodiments include sheaths, methods of forming a sheath, and devices that include a sheath and a needle disposed within it. Embodiments of the present disclosure may be implemented in an endoscopic system for collecting tissue samples at desired locations, such as in or in proximity of the gastrointestinal or pancreatic tract, where soft tissue samples are typically collected for diagnostic biopsy. Advantageously, embodiments of the present disclosure allow for effective collection of a desired amount of tissue sample at a desired location, thereby increasing the success rate and efficiency of collecting adequate tissue samples in an endoscopic procedure.

As described herein, an endoscope, such as an ultrasound endoscope, typically includes a proximal end, a distal (or “sensing”) end, and an internal working channel extending between the distal end and the proximal end. The term “proximal” (e.g., “a proximal end”) refers to a point or a location along the length of the endoscope that is closer to a physician or a medical practitioner, and the term “distal” (e.g., “a distal end”) refers to a point or location along the length of the endoscope that is closer to a sampling location in the body of a patient. A biopsy needle device is typically introduced into the working channel of the endoscope from the proximal end to the distal end of the endoscope until a distal end of the needle device approximates or reaches a desired location for collecting one or more tissue samples.

The biopsy channel of an endoscope is typically made of or lined with polytetrafluoroethylene (PTFE). The biopsy needle device includes a needle and a sheath, which encloses the needle and protects this channel from damage by the needle tip. The sheath may also serve to protect the patient, doctors, and assistants from inadvertent needle stick injuries and to guard the needle tip itself until the doctor deploys the needle from the distal end of the sheath to take a biopsy.

Fine needle biopsy (FNB) and fine needle aspiration (FNA) for use in endoscopic tissue collection are techniques that are commonly employed during Endoscopic Ultrasound (EUS) procedures. Typically, the sensing end of the endoscope has already been positioned proximate to the desired sampling location before the biopsy needle device is inserted into the biopsy channel of the endoscope (for example, because it is harder to navigate the sensing end once the needle is inserted). Once the sensing tip of the endoscope has been manipulated into the desired position, the process of taking a tissue sample may include (1) inserting the biopsy needle device into the endoscope biopsy channel until the distal end is next to the location to be sampled, (2) deploying the needle beyond the distal end of the sheath at least once to obtain the sample, and (3) withdrawing the biopsy needle device from the endoscope biopsy channel to collect the sample. This process may be repeated multiple times (i.e., to accumulate more sample tissue from essentially the same sampling location) while the sensing tip is maintained at the desired position.

The sheath should be flexible enough to allow the biopsy needle device to be deployed through the endoscope working channel, which is typically more than one meter long. In a typical endoscopic biopsy application, the sheath is approximately 57″ (fifty-seven inches) long. Such deployment may require the biopsy needle device to follow the bends and curves of the endoscope while in the channel, including in the bending section and while passing through the elevator mechanism (i.e., the mechanism which manipulates the sensing tip). In addition to such flexibility, however, it may also be desirable for the sheath to have enough column strength to permit the biopsy needle device to be pushed down the endoscope channel from the proximal end with minimum effort by the user, especially for a case in which the needle has a small gauge (e.g., 25- to 22-gauge).

Other desirable features for the sheath may include a resistance to deformation. A polymer sheath, for example, may tend to ovalize at a sharp bend, so that the inner diameter of the sheath decreases in a radial direction of the bend. Such deformation may cause the sheath to pinch the needle within it, increasing friction and restricting needle movement. A sheath that ‘takes a set’ or is otherwise slow to recover its original shape after being bent (e.g., by a bending section at a distal end of the channel) may guide the needle off course during needle puncture. It may be desirable for the sheath to allow for smooth advancement and retraction of the needle (e.g., under minimal and constant friction) with a minimum of effort by the user.

The desirable features noted above are often mutually exclusive. A sheath that has good column strength, for example, will typically lack flexibility. Flexible sheaths, on the other hand, tend to deform during use and/or may lack the ability to be advanced and/or retracted smoothly.

Multiple types of sheaths for FNA needles are currently available on the market. The most common type of sheath is a solid polymer tube (typically PEEK (polyether ether-ketone)). Another type of sheath is a closely wound coil spring. A further type of sheath is a braided polymer extrusion. Each type has characteristics that are desirable along with others that limit its performance.

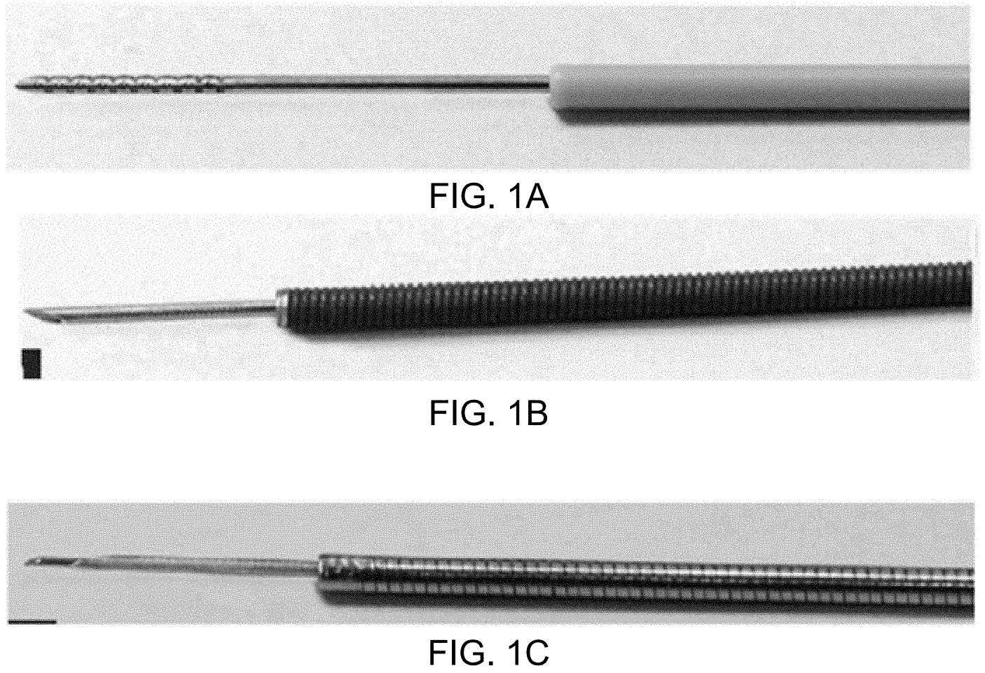

Traditional polymer sheaths tend to perform well in column strength. PEEK sheaths, for example, usually allow for smooth advancement and retraction. FIG. 1A shows an example of a PEEK tube. However, they typically have low flexibility and a tendency to deform during use. They can kink or ovalize during use, especially during endoscope biopsy channel insertion. Such deformation can create friction between the needle and the sheath and/or permanently damage the needle.

Sheaths which are closely wound helical coils of coated or uncoated wire (e.g., as shown in FIGS. 1B and 1C, respectively) are typically very flexible, have good column strength, and resist deformation. However, these sheaths may not perform well during retraction. If the endoscope is in a tortuous path, for example, a coil sheath will tend to stretch during retraction. Removing a needle with a coil sheath from an endoscope often requires the physician to straighten out the endoscope. If good visualization of the biopsy target has already been attained, such action is likely to be very undesirable, especially if multiple samples are to be taken from the same location.

Extruded polymer sheaths which have either a coil or braid inside are typically highly flexible. Unfortunately, such sheaths may deform in a tortuous path and also tend to stretch or otherwise deform during needle retraction. Such deformation may cause unpredictable behavior of the needle (e.g., jumping or stuttering) during retraction, such that the degree to which the surgeon retains direct control over the needle may vary over different stages of the retraction.

A sheath as disclosed herein includes a closely wound helical coil of metal wire (also called a “spring guide”), which provides flexibility and column strength, within a polymer (e.g., fluoropolymer) heat-shrink sleeve, which provides lubricity and tensile strength. Such a sheath may be implemented to be highly resistant to kinking, to show minimal deformation even when the bending section and elevator are used repeatedly, and to be unlikely to ovalize under the forces encountered during normal endoscopy. The metal coil may be implemented to create a barrier that is essentially impenetrable by the needle, protecting the endoscope biopsy channel, the patient, and the medical staff while minimizing friction on the needle.

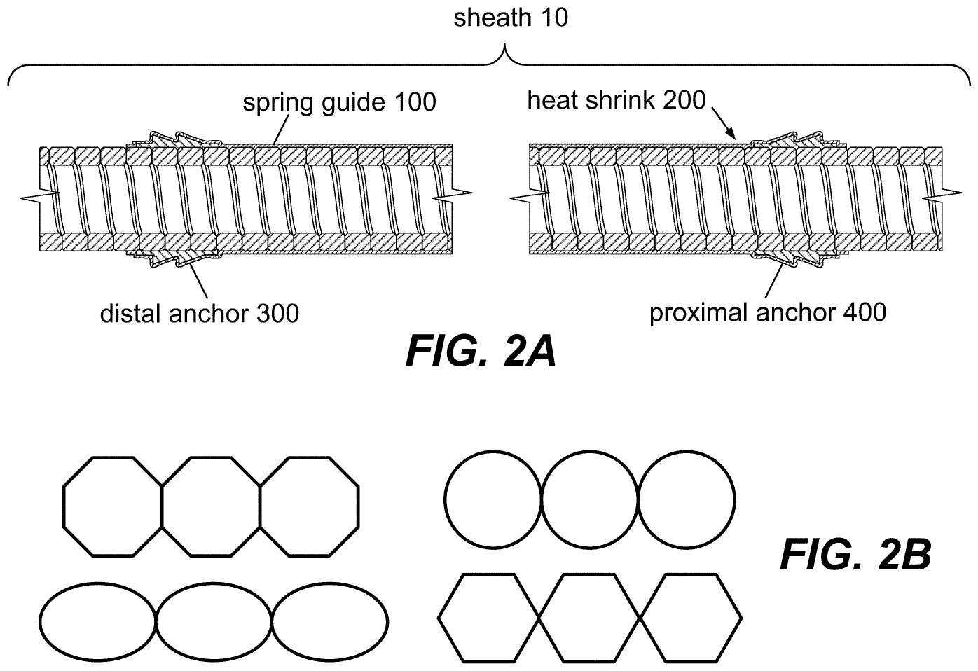

As shown in FIG. 2A, sheath 10 comprises a spring guide 100 (e.g., a stainless-steel coil spring guide), a sleeve of polymer heat-shrink tubing (PHS) 200, a distal anchor 300 near a distal end of the sheath, and a proximal anchor 400 near a proximal end of the sheath. Due to spring guide 100 that forms its backbone, sheath 10 may be implemented to have both column strength (for pushing the device into the endoscope channel) as well as flexibility (for navigating a tortuous path).

The length of sheath 10 may be one meter or more. For a typical endoscopic biopsy application, sheath 10 is approximately 57″ (fifty-seven inches) long; however, this length can be tuned longer or shorter to fit different applications by changing the length of spring guide 100 and PHS 200. Sheath 10 is typically combined with a needle that is movably disposed within the lumen of the sheath and is somewhat longer than the sheath, such that the needle can be manipulated at the proximal end to be deployed for sampling tissue at the distal end. The needle has a hollow tip for tissue collection and is typically made of a stainless-steel alloy and/or a nickel-titanium alloy (e.g., Nitinol). The outer surface of the needle may include surface features to increase echogenicity under ultrasound illumination. Typically the inner lumen of the needle is occupied by a stylet that is withdrawn at least partially (e.g., by manipulation at the proximal end of the sheath) before the sample is taken.

Spring guide 100 is typically made of a stainless-steel alloy (e.g., Society of Automotive Engineers (SAE) grade 304, 316, or 316L) but may also be made of other materials suitable for springs for use within the human body, such as a cobalt-chromium alloy. Due to the limited bioexposure of coil 100 in an endoscopic biopsy application, and especially with most or all of the exterior of coil 100 being covered by PHS 200, it may be possible to implement coil 100 using a material (e.g., another stainless-steel alloy) whose corrosion resistance and/or biocompatibility may be unsuitable for applications involving longer bioexposure (e.g., implantation).

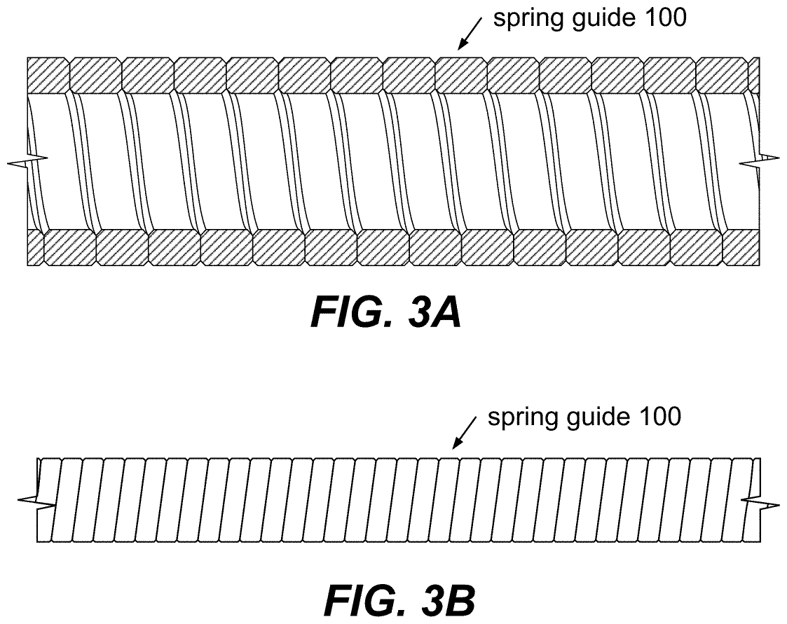

In the schematic examples illustrated in, e.g., FIGS. 2A and 3A, the spring coil is composed of flat wire stock that is closely wound into a helix to define an interior space (i.e., a lumen) such that the inner diameter of the coil is uniform along its length. In the particular example of spring guide 100 as shown in cross-section in FIG. 3A, the flat wire stock has a ratio of width to thickness of about 3:2. In one such example, the flat wire stock is 0.020″ (twenty one-thousandths of an inch) wide and 0.014″ (fourteen one-thousandths of an inch) thick, and the outer diameter of spring guide 100 is about 0.080″ (eighty one-thousandths of an inch). Each of these dimensions may be increased or decreased depending on the specific application, and coil thickness, shape, diameter and/or winding tension may also be varied to tune column strength, flexibility and/or deformability.

The flat wire stock in these examples has a cross-section that is at least substantially rectangular (i.e., having flat surfaces that are parallel or orthogonal to each other, and corners that may be rounded). In other examples, the wire stock may be round, square or pressed into any number of shapes in different diameters, thickness and widths to tune flexibility and column strength. As shown in FIG. 2B, for example, the cross-section of the wire stock may be at least substantially polygonal (e.g., hexagonal or octagonal), at least substantially elliptical (i.e., may be flattened along the major and/or minor axes), or at least substantially circular (i.e., may be flattened along a direction parallel to a longitudinal axis of the coil and/or a direction orthogonal to that axis).

A potential advantage of using flat wire to form coil 100 is that flat wire can provide good column strength in a small-diameter package. For example, a flat-wire coil that defines a lumen of a particular diameter has a smaller outer diameter than a coil made of round wire having the same cross-sectional area as the flat wire. Another potential advantage of flat wire over round wire is that the slight depressions between adjacent windings of a flat-wire implementation of spring guide 100 allow PHS 200 to grip the spring guide enough to anchor itself, but not enough to creep between the windings upon shrinking as could occur with a round wire. Such creep could result in a sheath that could more easily plastically deform when bent. Under similar parameters, therefore, a round-wire spring guide may result in a more plastically deformable sheath than a flat-wire spring guide.

Spring guides can be excellent under compression but may perform poorly under tension, so the addition of the PHS 200 on top of the spring guide creates a tension-resistant cover that resists stretching during needle retraction and device removal. It may be desirable to use a PHS made of fluoropolymer, as such materials tend to exhibit high tensile strength at a suitably small thickness and to have very high lubricity, which may be desirable for navigating the sheath down an endoscope channel. The fluoropolymer FEP (fluorinated ethylene propylene) is a particular example of a fluoropolymer that may be used for PHS 200. Other materials, including PET (polyethylene terephthalate) and PEBAX (polyether block amide, a thermoplastic elastomer), may also be used but have been found to be less optimal than a fluoropolymer, and specifically less optimal than FEP. Under similar dimensional constraints, PET was found to provide sufficient stiffness but suboptimal flexibility, and PEBAX was found to provide suboptimal tensile strength.

It may be desirable to implement PHS 200 using a tubing whose outer diameter will shrink by about thirty to forty percent. A fluoropolymer tubing (e.g., FEP) can provide such a characteristic, as opposed to polyolefin heat-shrink, for example, whose outer diameter may shrink by fifty to sixty percent or more. It may be desirable for the outer diameter of PHS 200, if permitted to fully shrink, to be less than the outer diameter of spring guide 100. Such relative dimensions may produce a gripping effect as discussed above.

For a case in which the outer diameter of spring guide 100 is about 0.080″ (eighty one-thousandths of an inch), it may be desirable to implement PHS 200 so that the approximate outer diameter of PHS 200 as shrunken over the spring guide is 0.092″ (ninety-two one-thousandths of an inch). In one such example, PHS 200 has an original outer diameter and thickness of about 0.092″ (ninety-two one-thousandths of an inch) and about 0.002″-0.003″ (two to three one-thousandths of an inch), respectively; a thickness of about 0.006″ (six one-thousandths of an inch) when shrunken over the spring guide; and a change in length due to shrinking of about minus one to minus two percent. In this example, the decrease in outer diameter due to shrinking is approximately balanced by an increase in outer diameter due to increased thickness upon shrinking.

The outer diameter and thickness of PHS 200 when permitted to fully shrink may be selected according to the tensile strength needed and the stiffness required. In the particular example described above, PHS 200 may be selected to have an outer diameter when permitted to fully shrink of about 0.060″ (sixty one-thousandths of an inch) and a thickness of about 0.010″ (ten one-thousandths of an inch) when fully shrunk. A thicker heat-shrink can provide greater tensile strength but may also increase stiffness and increase overall sheath diameter.

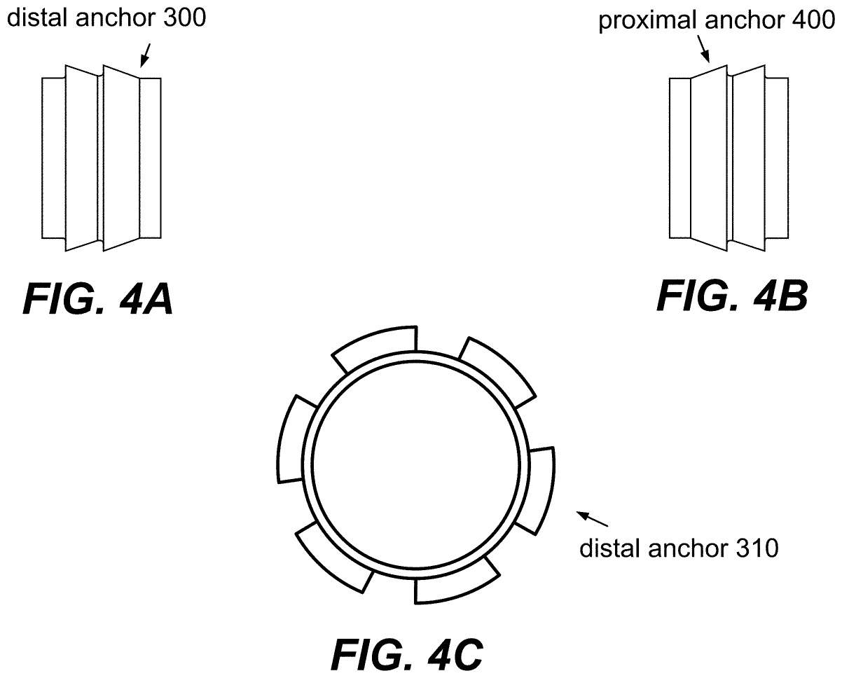

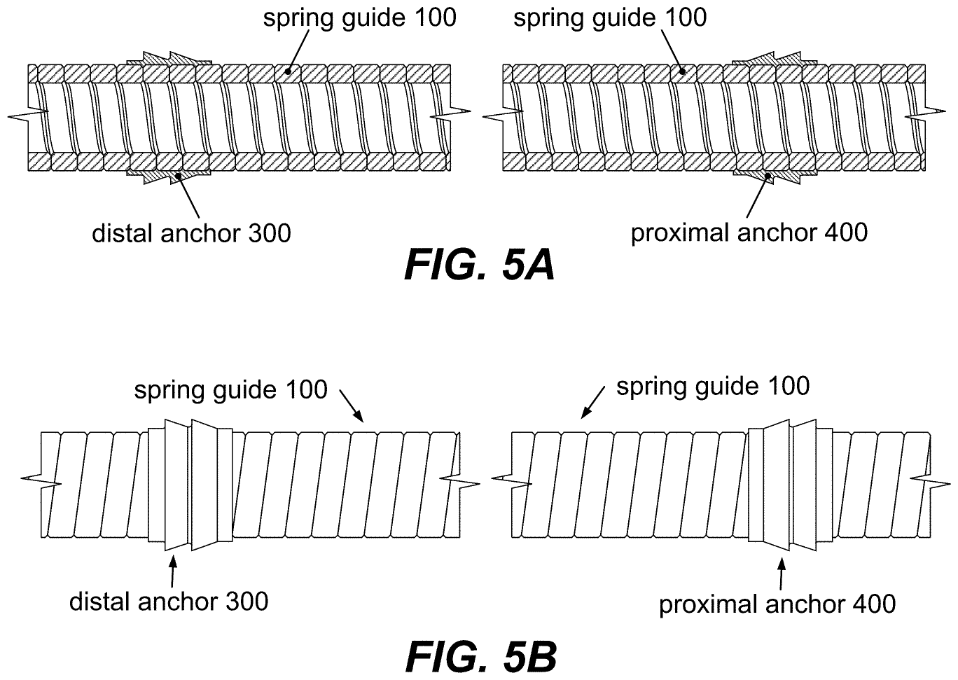

Distal anchor 300 is secured to spring guide 100 near a distal end of sheath 10, and proximal anchor 400 is secured to spring guide 100 near a proximal end of sheath 10. FIGS. 4A and 4B show side views of distal anchor 300 and proximal anchor 400, respectively. Anchors 300 and 400 are secured to the outer surface of spring guide 100, preferably by welding. Less preferred alternatives to welding include other methods of affixing, such as adhesion (e.g., gluing), and methods of compression fit, such as crimping or an interference fit (e.g., by heating the anchor to temporarily expand its inner diameter immediately before insertion of coil 100 through that inner diameter). FIGS. 5A and 5B show cross-sectional and side views, respectively, of distal anchor 300 and proximal anchor 400 secured to spring guide 100.

Each of anchors 300 and 400 includes at least one protrusion (also described herein as a “barb”) that extends away from the axis of the anchor and over which PHS 200 is shrunk. The protrusions provide anchoring points that serve to grip PHS 200 when it is shrunk, inhibiting movement (e.g., slipping or creeping) of PHS 200 along the spring guide during device use and thus maintaining the tensile strength of the sheath. The anchor may include, for example, two or more protrusions that are regularly spaced around the circumference of the anchor. FIG. 4C shows an end view of one such example 310 of distal anchor 300 that includes six barbs evenly distributed around the circumference of the anchor, each one having an angular width of about thirty degrees. Alternatively, a protrusion may encircle the anchor (as shown, e.g., in FIGS. 4A, 4B, and 5B).

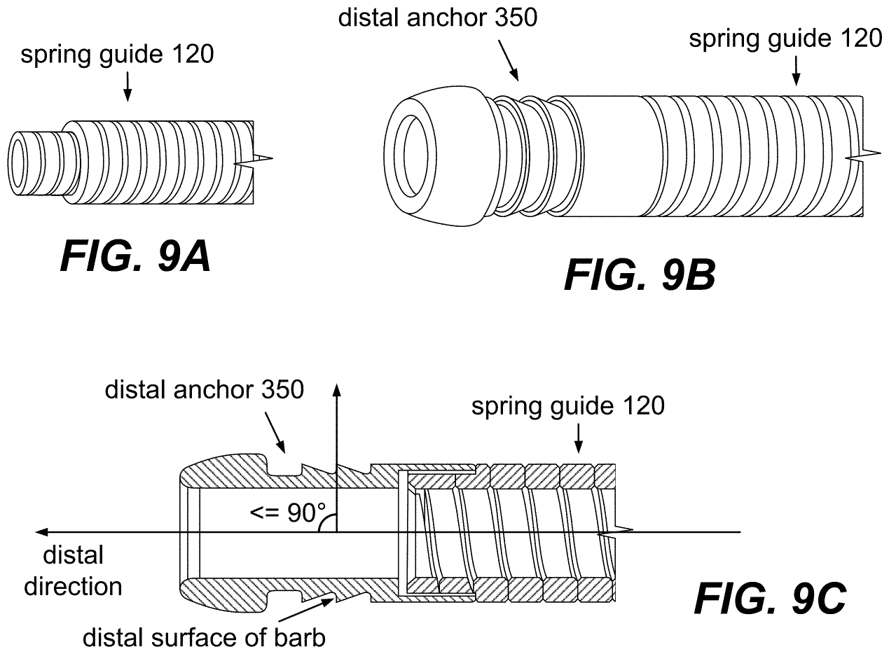

Each protrusion is implemented to have a surface which faces the closest end of the sheath, as determined when the anchor is secured to coil 100, and which forms an angle of not more than ninety degrees with the axis of the coil in the direction of the closest end of the sheath, as demonstrated in FIG. 9C. Typically each protrusion is also implemented to have another (outer) surface that is inclined away from the outer surface of the sheath, along a direction that is parallel to the longitudinal axis of coil 100 and toward the closest end of the sheath.

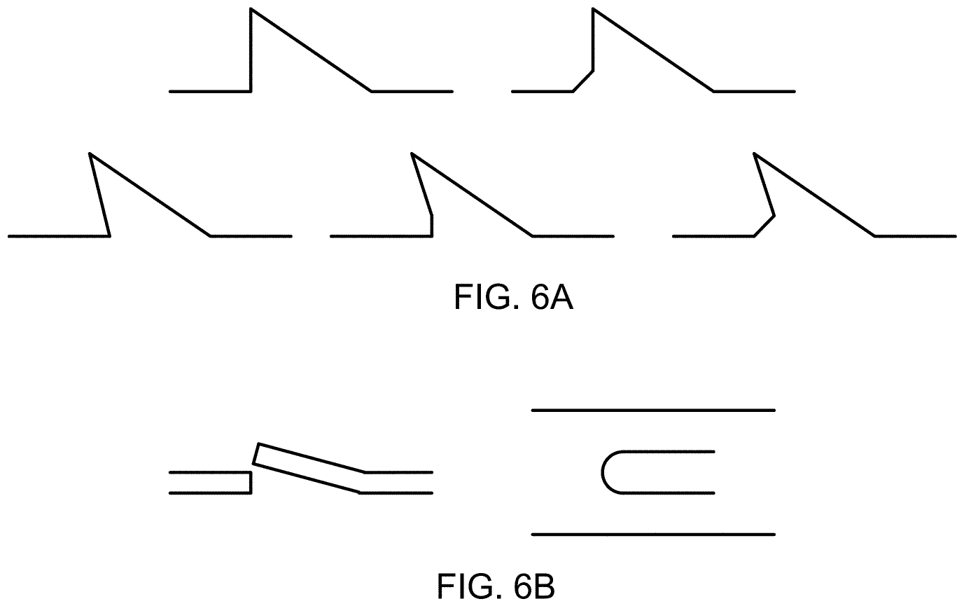

FIG. 6A shows profiles of other examples of a barb of anchor 300 and/or 400, and FIG. 6B shows a cross-section (left) and a plan view (right) of a further example of such a barb. It will be understood that the sharp points and corners shown in these diagrams will be somewhat rounded in actual practice. It may be desirable, for example, to avoid puncturing the PHS as it is shrunk over the points of the barb.

The height of each protrusion (i.e., radial distance from tip to base of the protrusion) may be selected, depending on PHS 200, such that the outer surface of PHS 200 remains relatively smooth after PHS 200 is shrunk over the protrusion or protrusions. For the particular example in which PHS 200 has a thickness of about 0.006″ (six one-thousandths of an inch) after shrinking, it may be desirable to implement each protrusion to have a height of 0.007″-0.008″ (seven to eight one-thousandths of an inch). In general, the height of the protrusion may be up to one, one-and-one-half, or two times the thickness of PHS 200 after shrinking.

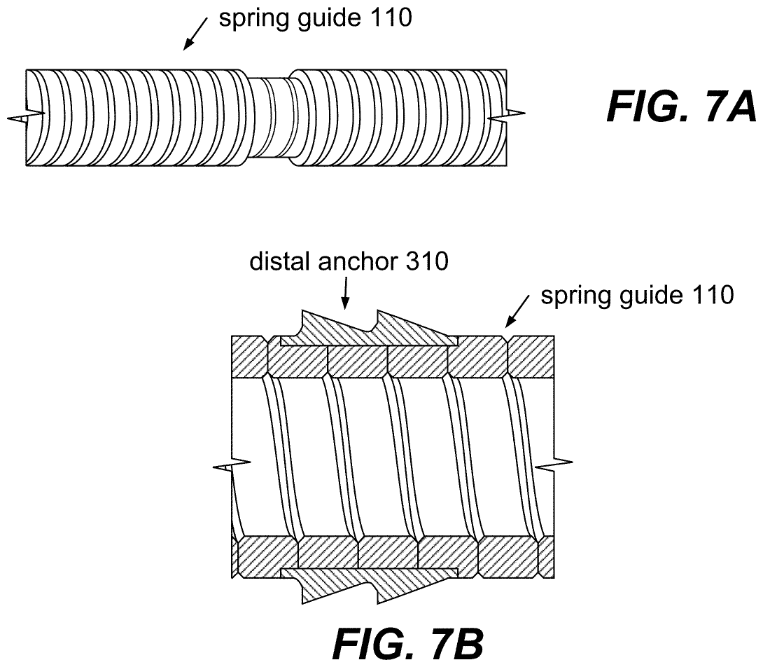

It may be desirable to minimize the extent to which the anchors may extend beyond the outer surface of coil 100. FIG. 7A shows a perspective view of an implementation 110 of coil 100 in which a recess to accommodate an implementation of distal anchor 300 or proximal anchor 400 has been ground circumferentially into an intermediate portion of the coil. Such a configuration may allow the maximum outer diameter of sheath 10 to be minimized. FIG. 7B shows a cross-sectional view of an implementation 310 of distal anchor 300 secured in the recess of spring guide 110. In one example, mounting of anchor 310 to coil 110 includes heating anchor 310 to temporarily expand it, and/or cooling coil 110 to temporarily contract it, so that anchor 310 may be slid into position over the recess.

PHS 200 extends at least slightly beyond the endmost protrusion of each anchor, such that PHS 200 may grip this protrusion effectively. Sheath 10 may also be implemented to include a retention ring (e.g., a collar), at one or both anchors, that encircles PHS 200 over the protrusion or protrusions, between adjacent protrusions, and/or between a protrusion and the respective end of PHS 200. Such a ring may be configured to compress PHS 200 and/or may include teeth or another gripping feature or texture on its inner surface.

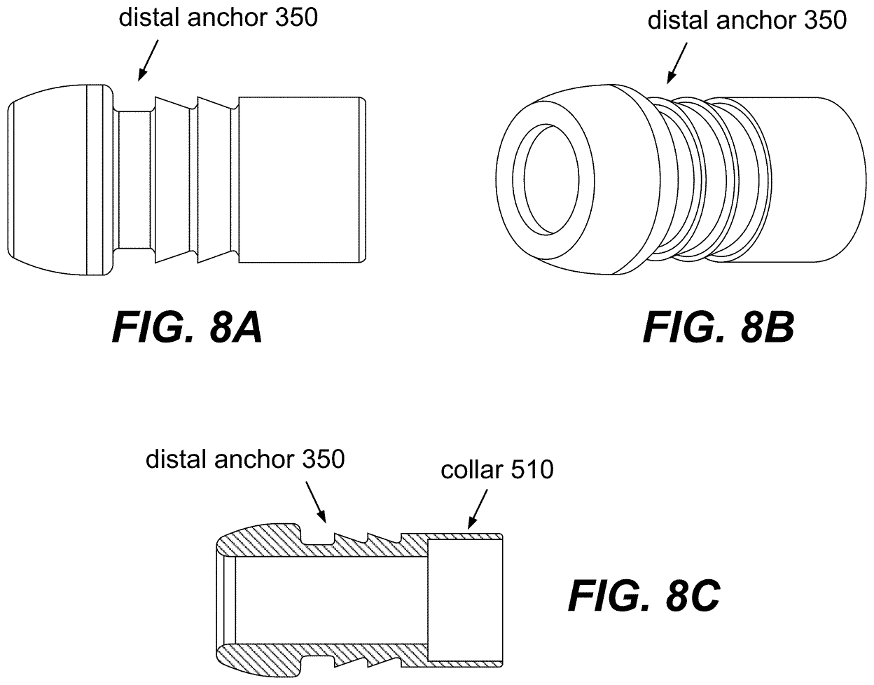

One or both of distal anchor 300 and proximal anchor 400 may be secured to coil 100 at the respective end of the coil. FIGS. 8A, 8B, and 8C show a side view, a perspective view, and a cross-sectional view, respectively, of an implementation 350 of distal anchor 300 that includes a distal tip (made, e.g., of stainless steel) that is smooth and atraumatic. In a typical implementation of a sheath 10 that includes distal anchor 350, a needle may be deployed from the distal atraumatic tip. Distal anchor 350 also includes a section (e.g., a collar 510) that can be secured (e.g., welded) to the coil 100. It may be desirable for the lumen of distal anchor 350 to have the same diameter as the lumen of coil 100.

Distal anchor 350 may be dimensioned so that the inner diameter of collar 510 is at least approximately equal (e.g., within a typical assembly tolerance) to the outer diameter of coil 100, such that the distal end of coil 100 may be received within collar 510. Alternatively, anchor 350 may be dimensioned so that the outer diameter of collar 510 is at least approximately equal to the outer diameter of coil 100. FIG. 9A shows a perspective view of an implementation 120 of coil 100 in which an end portion has been ground down circumferentially so that it may be secured within the collar 510 of such an implementation of anchor 350. Such a configuration may allow the maximum outer diameter of sheath 10 to be minimized. FIGS. 9B and 9C show a perspective view and a cross-sectional view, respectively, of distal anchor 350 mounted to the end of spring guide 120.

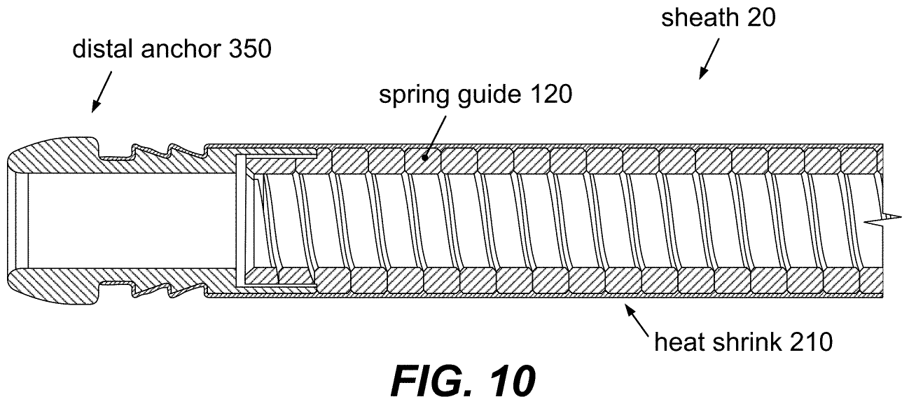

FIG. 10 shows a cross-sectional view of an implementation 20 of sheath 10 that includes distal anchor 350, spring guide 120, and an implementation 210 of PHS 200 that extends to the atraumatic tip. It may be seen that distal anchor 350 includes two barbs which each encircle the anchor, and the distal lip of the collar of anchor 350 may also provide purchase to PHS 210. It will also be noted that the bases of the protrusions of distal anchor 350 are closer to the central axis of coil 120 than the outer surface of coil 120 is, such that sheath 20 may have a smaller outer diameter above the protrusions than sheath 10, for the same radial dimensions of coil 100 and PHS 200.

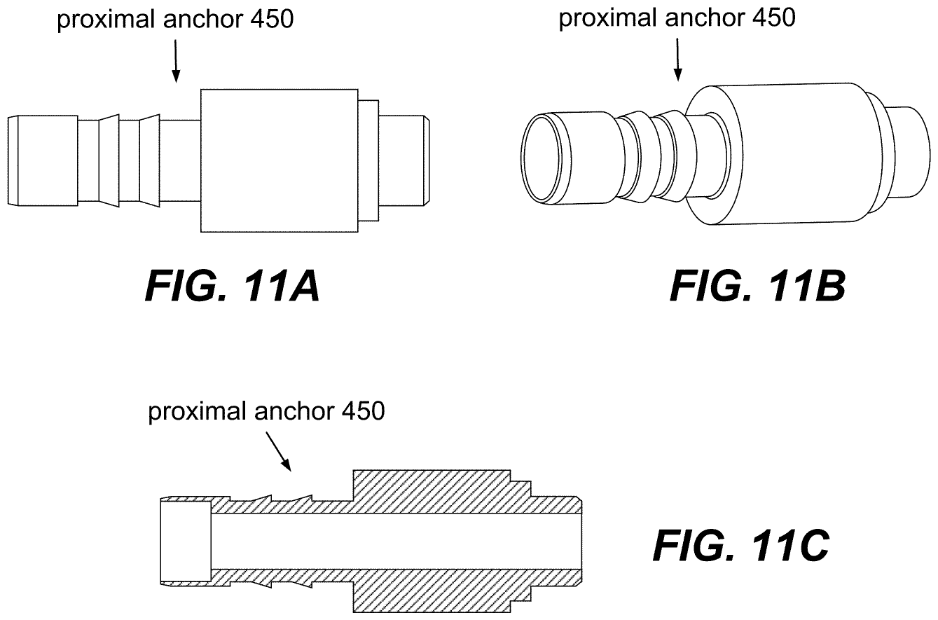

FIGS. 11A, 11B, and 11C show a side view, a perspective view, and a cross-sectional view, respectively, of an implementation 450 of proximal anchor 400 that includes a proximal tip (made, e.g., of stainless steel). This proximal tip may be shaped to engage a feature in the handle of the endoscope. The proximal end of anchor 450 may be configured, for example, to be attached to a hub of a handle from which the sheath is inserted into the endoscope biopsy channel and with which the physician controls deployment of the needle beyond the distal end of the sheath. Additionally or alternatively, the proximal end of anchor 450 may be configured to prevent the needle from being fully withdrawn from the sheath, or sheath 10 may be otherwise implemented to include a needle that is permanently mounted to the sheath.

Like distal anchor 350, proximal anchor 450 includes a section (e.g., a collar) that can be secured (e.g., welded) to the coil 100. In one example, the inner diameter of the collar is at least approximately equal to the outer diameter of coil 100. It may be desirable for the lumen of proximal anchor 450 to have the same diameter as the lumen of coil 100. It may be seen in FIGS. 11A, 11B, and 11C that proximal anchor 450 includes two barbs which each encircle the anchor, and the proximal lip of the collar of anchor 450 may also provide purchase to PHS 200.

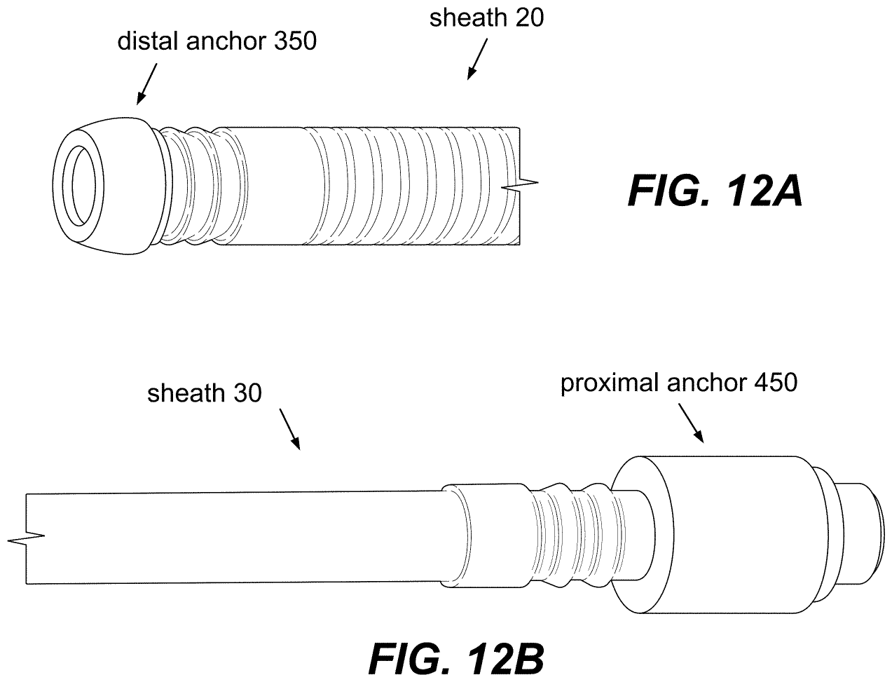

FIG. 12A shows a perspective view of a distal end of sheath 20. This figure illustrates that a minimum outer diameter of PHS 210 over distal anchor 350 is less than an outer diameter of PHS 210 over coil 120. FEP is typically transparent, and the individual windings of coil 120 are also visible in this figure. In some cases, a slight shrinking of PHS 210 into depressions between adjacent windings of coil 120 may also be discernible and/or palpable at the outer surface of PHS 210.

While FEP is typically transparent, in practice PHS 200 may have any color or degree of transparency so long as such coloring does not excessively reduce its tensile strength or render it unsuitable (e.g., too thin or thick) upon shrinking. FIG. 12B shows a perspective view of a proximal end of an implementation 30 of sheath 20 that includes an opaque implementation of PHS 210 which extends beyond the most proximal protrusion of proximal anchor 450. Sheath 30 includes an instance of coil 120 whose proximal end is secured within the collar of proximal anchor 450.

The principles described herein may be practiced as described to obtain implementations of sheath 10, and biopsy needle devices including such implementations, that provide advantages such as protecting the endoscope biopsy channel from needle damage; protecting the patient, doctors, and assistants from inadvertent needle stick injuries; guarding the needle until the doctor deploys the needle for taking a biopsy; having enough column strength to be pushed down the endoscope channel from the proximal end with a minimum of effort by the user; having sufficient flexibility to be deployed through the endoscope working channel and follow the bends and curves of the endoscope while in the channel, including in the bending section and while passing through the elevator mechanism; resisting a degree of deformation that might cause the sheath to guide the needle off course during needle puncture; and/or consistently allowing for smooth advancement and retraction of the needle with a minimum of effort by the user (e.g., minimal friction) regardless of device and endoscope orientation.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A sheath comprising:
a coil of wire, the coil having an interior that defines a lumen;
a distal anchor secured to the coil near a distal end of the coil and having a barb that extends away from an axis of the coil;
a proximal anchor secured to the coil near a proximal end of the coil and having a barb that extends away from the axis of the coil; and
a polymer heat-shrink sleeve that is coaxial with the coil, circumferentially encloses and is shrunken onto the coil over a length from the distal anchor to the proximal anchor, and extends over the barb of the distal anchor and the barb of the proximal anchor,
wherein, in a first plane that includes the axis of the coil, an angle between a distal direction of the axis and a distal surface of the barb of the distal anchor is not more than ninety degrees, and
wherein, in a second plane that includes the axis of the coil, an angle between a proximal direction of the axis and a proximal surface of the barb of the proximal anchor is not more than ninety degrees.

2. The sheath according to claim 1, wherein a distal end of the sleeve is closer to a distal end of the sheath than the barb of the distal anchor is, and wherein a proximal end of the sleeve is closer to a proximal end of the sheath than the barb of the proximal anchor is.

3. The sheath according to claim 1, wherein, for at least one among the distal anchor and the proximal anchor, the distal surface of the barb extends at least thirty degrees around a circumference of the coil.

4. The sheath according to claim 1, wherein, for at least one among the distal anchor and the proximal anchor, the distal surface of the barb encircles the coil.

5. The sheath according to claim 1, wherein the distal anchor has a second barb that extends away from an axis of the coil, and wherein the sleeve extends over the second barb of the distal anchor.

6. The sheath according to claim 1, wherein an outer diameter of the coil is substantially constant over the length from the distal anchor to the proximal anchor, and
wherein an inner diameter of the sleeve, in at least one plane that is orthogonal to the axis of the coil and intersects the distal anchor, is less than the outer diameter of the coil.

7. The sheath according to claim 1, wherein an outer diameter of the coil is substantially constant over the length from the distal anchor to the proximal anchor, and
wherein an outer diameter of the distal anchor, in at least one plane that is orthogonal to the axis of the coil, is less than the outer diameter of the coil.

8. The sheath according to claim 1, wherein a part of the distal anchor that is circumferentially enclosed by the sleeve has an outer diameter that is not greater than an outer diameter of the coil.

9. The sheath according to claim 1, wherein the distal anchor includes an atraumatic tip.

10. The sheath according to claim 1, wherein the distal anchor forms a distal end of the sheath.

11. The sheath according to claim 1, wherein the sleeve is made of a fluoropolymer.

12. A biopsy needle device, the device including:
a sheath comprising:
a coil of wire, the coil having an interior that defines a lumen;
a distal anchor secured to the coil near a distal end of the coil and having a barb that extends away from an axis of the coil;
a proximal anchor secured to the coil near a proximal end of the coil and having a barb that extends away from the axis of the coil; and
a polymer heat-shrink sleeve that is coaxial with the coil, circumferentially encloses and is shrunken onto the coil over a length from the distal anchor to the proximal anchor, and extends over the barb of the distal anchor and the barb of the proximal anchor; and
a needle moveably disposed within the lumen,
wherein, in a first plane that includes the axis of the coil, an angle between a distal direction of the axis and a distal surface of the barb of the distal anchor is not more than ninety degrees, and
wherein, in a second plane that includes the axis of the coil, an angle between a proximal direction of the axis and a proximal surface of the barb of the proximal anchor is not more than ninety degrees.

13. The biopsy needle device according to claim 12, wherein the needle has echogenic surface features.

14. The biopsy needle device according to claim 12, wherein the device comprises a stylet moveably disposed within the needle.

15. The biopsy needle device according to claim 12, wherein the needle comprises a distal portion made of an alloy principally comprising nickel and titanium and a proximal portion made of a stainless steel alloy.

16. The biopsy needle device according to claim 12, wherein a length of the device is at least one meter.

17. A method of forming a sheath, the method comprising:
securing a distal anchor near a distal end of a coil of wire, the coil having an interior that defines a lumen, the distal anchor having a barb that extends away from an axis of the coil;
securing a proximal anchor near a proximal end of the coil, the proximal anchor having a barb that extends away from the axis of the coil; and
applying heat to shrink a polymer heat-shrink sleeve to be coaxial with and shrunken onto the coil,
wherein the shrunken sleeve circumferentially encloses the coil over a length from the distal anchor to the proximal anchor and extends over the barb of the distal anchor and the barb of the proximal anchor, and
wherein, in a first plane that includes the axis of the coil, an angle between a distal direction of the axis and a distal surface of the barb of the distal anchor is not more than ninety degrees, and
wherein, in a second plane that includes the axis of the coil, an angle between a proximal direction of the axis and a proximal surface of the barb of the proximal anchor is not more than ninety degrees.

18. The method according to claim 17, wherein the distal anchor has a second barb that extends away from an axis of the coil, and wherein the sleeve extends over the second barb of the distal anchor.

19. The method according to claim 17, wherein an outer diameter of the coil is substantially constant over the length from the distal anchor to the proximal anchor, and
wherein an inner diameter of the sleeve, in at least one plane that is orthogonal to the axis of the coil and intersects the distal anchor, is less than the outer diameter of the coil.

20. The method according to claim 17, wherein a part of the distal anchor that is circumferentially enclosed by the sleeve has an outer diameter that is not greater than an outer diameter of the coil.

21. The method according to claim 17, wherein the method comprises deploying a needle within the sheath, and wherein a length of each of the needle and the sheath is at least one meter.
